(19) United States
(12) Patent Application Publication
(10) Pub. No.: US 2010/0240591 A1
(43) Pub. Date: Sep. 23, 2010

(54) TPP II INHIBITORS FOR USE IN THE TREATMENT OF AUTOIMMUNE AND INFLAMMATORY DISEASES AND TRANSPLANT REJECTION

(75) Inventors: Rickard Glas, Stockholm (SE); Hong Xu, Taby (SE)

Correspondence Address: BROOKS KUSHMAN P.C., 1000 TOWN CENTER, TWENTY-SECOND FLOOR, SOUTHFIELD, MI, 48075, US

(73) Assignee: ONCOREG AB, Stockholm (SE)

(21) Appl. No.: 12/666,457

(22) PCT Filed: Jun. 25, 2007
(86) PCT No.: PCT/EP07/05620

§ 371 Date: May 20, 2010

(51) Int. Cl. A61K 38/06; G01N 33/566; A61P 37/06; A61P 1/00

(52) U.S. Cl. 514/13.2; 514/21.9; 514/16.6; 514/18.7; 436/501

(57) ABSTRACT

TPP II (tripeptidyl peptidase II) inhibitors are useful in the treatment of autoimmune and/or inflammatory diseases, for example Systemic Lupus Erythematosus, Rheumatoid Arthritis, Multiple Sclerosis, Sjögrens Syndrome, Diabetes Mellitus Type I or II, Psoriasis, Eczema, Ulcerous Colitis, and Chron's Disease, or transplant rejection. Suitable compounds comprise tripeptide compounds of general formula R^{N1}, R^{N2}, A¹, A², A³ and R^{C1} are as defined herein, and which include for example the tripeptide sequences GLA and GPG.

The present invention relates to the use of compounds in the treatment of autoimmune and inflammatory diseases and transplant rejection.

Many detailed studies have been carried out on tripeptidyl-peptidase II (TPP II). TPPII is built from a unique 138 kDa sub-unit expressed in multi-cellular organisms from Drosophila to Homo Sapiens (Tomkinson B, Lindas A C. Tripeptidyl-peptidase II: a multi-purpose peptidase. Int J Biochem Cell Biol 2005; 37:1933-7) (Renn S C, Tomkinson B, Taghert P H. Characterization and cloning of tripeptidyl peptidase II from the fruit fly, Drosophila melanogaster. J Biol Chem 1998; 273:19173-82) (Rockel B, Peters J, Kuhlmorgen B, Glaeser R M, Baumeister W. A giant protease with a twist: the TPPII complex from Drosophila studied by electron microscopy. EMBO J. 2002; 21:5979-84). Data from Drosophila suggests that the TPPII complex consists of repeated sub-units forming two twisted strands with a native structure of about 6 MDa (Rockel B, Peters J, Kuhlmorgen B, Glaeser R M, Baumeister W. A giant protease with a twist: the TPPII complex from Drosophila studied by electron microscopy. EMBO J. 2002; 21:5979-84). TPPII degrades cytosolic polypeptides (Glas R, Bogyo M, McMaster J S, Gaczynska M, Ploegh H L. A proteolytic system that compensates for loss of proteasome function. Nature 1998; 392:618-22) (Geier E, Pfeifer G, Wilm M, Lucchiari-Hartz M, Baumeister W, Eichmann K, et. al. A giant protease with potential to substitute for some functions of the proteasome. Science 1999; 283:978-81) (Gavioli R, Frisan T, Vertuani S, Bornkamm G W, Masucci M G. c-myc overexpression activates alternative pathways for intracellular proteolysis in lymphoma cells. Nat Cell Biol 2001; 3:283-8.), generates certain MHC class I ligands (Reits E, Neijssen J, Herberts C, Benckhuijsen W, Janssen L, Drijfhout J W, et. al. A major role for TPPII in trimming proteasomal degradation products for MHC class I antigen presentation. Immunity 2004; 20:495-506) (York I A, Bhutani N, Zendzian S, Goldberg A L, Rock K L. Tripeptidyl Peptidase II Is the Major Peptidase Needed to Trim Long Antigenic Precursors, but Is Not Required for Most MHC Class I Antigen Presentation. J Immunol 2006; 177:1434-43.) and complements the proteasome in protein turnover. However, other roles of this complex may also exist, that may be unrelated to protein turnover. TPPII regulates transduction of apoptotic signals as well as centrosome homeostasis, by unclear mechanisms (Hong X, Lei L, Glas R. Tumors acquire inhibitor of apoptosis protein (IAP)-mediated apoptosis resistance through altered specificity of cytosolic proteolysis. J Exp Med 2003; 197:1731-43.) (Hilbi H, Puro R J, Zychlinsky A. Tripeptidyl peptidase II promotes maturation of caspase-1 in Shigella flexneri-induced macrophage apoptosis. Infect Immun 2000; 68:5502-8) (Stavropoulou V, Xie J, Henriksson M, Tomkinson B, Imreh S, Masucci MG. Mitotic infidelity and centrosome duplication errors in cells overexpressing tripeptidyl-peptidase II. Cancer Res 2005; 65:1361-8) (Stavropoulou V, Vasquez V, Cereser B, Freda E, Masucci M G. TPPII promotes genetic instability by allowing the escape from apoptosis of cells with activated mitotic checkpoints. Biochem Biophys Res Commun 2006; 346:415-25).

Whilst many scientists have carried out work on many aspects of the above-mentioned pathways, it has not hitherto been recognized that TPP II inhibitors can be used to treat autoimmune or inflammatory diseases or transplant rejection.

From a first aspect the present invention provides a compound for use in the treatment of an autoimmune or inflammatory disease or transplant rejection, wherein said compound is a TPP II inhibitor.

As used herein the term treatment covers the treatment of an established autoimmune or inflammatory condition or transplant rejection state, or diseases that are consequences thereof, as well as preventative therapy and the treatment of a pre-autoimmune, pre-inflammatory or pre-rejection condition.

The conditions of autoimmunity, inflammation, and transplant rejection may exist separately, or two or all three of them may be present at the same time. Commonly, for example, conditions of an autoimmune origin may also result in, or be associated with, inflammation.

From a further aspect the present invention provides a compound for use in the treatment of an autoimmune or inflammatory disease or transplant rejection, wherein said compound is selected from the following formula (i) or is a pharmaceutically acceptable salt thereof:

R^{N1}R^{N2}N-A¹-A²-A³-CO—R^{C1} (i)
wherein A¹, A² and A³ are amino acid residues having the following definitions according to the standard one-letter abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,
R^{N1} and R^{N2} are each attached to the N terminus of the peptide, are the same or different, and are each independently
   R^{N3},
   (linked)-R^{N3},
   CO-(linker1)-R^{N3},
   CO—O-(linker1)-R^{N3},
   CO—N-((linker1)-R^{N3})R^{N4} or
   SO₂—(linker1)-R^{N3},
(linker1) may be absent, i.e. a single bond, or CH₂, CH₂CH₂, CH₂CH₂CH₂, CH₂CH₂CH₂CH₂ or CH═CH,
R^{N3} and R^{N4} are the same or different and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
   wherein there may be zero, one or two (same or different) optional
   substituents on R^{N3} and/or R^{N4} which may be:
   hydroxy-;
   thio-:
   amino-;
   carboxylic acid;
   saturated or unsaturated, branched or unbranched C₁₋₆ alkyloxy;
   saturated or unsaturated, branched or unbranched C₃₋₁₂ cycloalkyl;
   N-, O-, or S-acetyl;
   carboxylic acid saturated or unsaturated, branched or unbranched C₁₋₆ alkyl ester;
   carboxylic acid saturated or unsaturated, branched or unbranched C₃₋₁₂ cycloalkyl ester
   phenyl;
   mono- or bicyclic C₁₋₁₀ heteroaryl;
   non-aromatic C₁₋₁₀ heterocyclyl; or
   halogen;
R^{C1} is attached to the C terminus of the tripeptide, and is:
   O—R^{C2},
   O-(linker2)-R^{C2},
   N((linker2)R^{C2})R^{C3}, or
   N(linker2)R^{C2}—NR^{C3}R^{C4},
(linker2) may be absent, i.e. a single bond, or C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2}, R^{C3} and R^{C4} are the same or different, and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
   wherein there may be zero, one or two (same or different) optional substituents on each of R^{C2} and/or R^{C3} and/or R^{C4} which may be one or more of:
      hydroxy-;
      thio-:
      amino-;
      carboxylic acid;
      saturated or unsaturated, branched or unbranched C₁₋₆ alkyloxy;
      saturated or unsaturated, branched or unbranched C₃₋₁₂ cycloalkyl;
      N-, O-, or S-acetyl;
      carboxylic acid saturated or unsaturated, branched or unbranched C₁₋₆ alkyl ester;
      carboxylic acid saturated or unsaturated, branched or unbranched C₃₋₁₂ cycloalkyl ester
      phenyl;
      halogen;
      mono- or bicyclic C₁₋₁₀ heteroaryl; or
      non-aromatic C₁₋₁₀ heterocyclyl.

The N and CO indicated in the general formula for formula (i) are the nitrogen atom of amino acid residue A¹ and the carbonyl group of amino acid residue A³ respectively.

From a further aspect the invention provides a method of treatment of an autoimmune or inflammatory disease or transplant rejection comprising administering to a patient in need thereof a therapeutically effective amount of a TPPII inhibitor or a compound selected from formula (i) or a pharmaceutically acceptable salt thereof.

Similarly, from a further aspect the present invention provides the use of a TPPII inhibitor or a compound selected from formula (i) or a pharmaceutically acceptable salt thereof in the manufacture of a medicament for the treatment of an autoimmune or inflammatory disease or transplant rejection.

Without wishing to be bound by theory, the invention may be considered to recognize that TPP II inhibitors are useful in the treatment of an autoimmune or inflammatory disease or transplant rejection.

Without wishing to be bound by theory, the efficacy of the present invention is believed to be a consequence of the link between TPP II inhibition and the PI3K/Akt pathway.

Autoimmune or inflammatory diseases are potential indications for inhibitors of the PI3K/Akt pathway. Activation of Akt kinase is one important component in signal transduction from growth factor receptors. Phosphorylation of Akt kinase at Ser473 induces its full activation, subsequent to Thr308 phosphorylation performed by PDK1 at the cell membrane (Patel R K, Mohan C. PI3K/AKT signaling and systemic autoimmunity. Immunol Res. 2005; 31(1):47-55). Akt phosphorylation at Ser473 is reported to require mTOR signaling, although several other PI3K-like kinases may also be involved. Our data have indicated that the expression of TPPII is controlled by mTOR, (mammalian target of Rapamycin), a member of the PI3K-family of kinases that integrates signals from nutrient sensing and growth factor receptor pathways (Wullschleger S, Loewith R, Hall M N. TOR signaling in growth and metabolism. Cell. 2006 Feb. 10; 124(3):471-84). Rapamycin is used in patients to inhibit immune responses, e.g. following transplantation. mTOR controls several important downstream targets that decide the outcome of growth factor signaling, such as the activation of Akt, a crucial kinase both in tumor biology and immunology. More specifically, signaling by receptors downstream of T cell receptor (TCR) ligation requires activation of Akt (Patel R K, Mohan C. PI3K/AKT signaling and systemic autoimmunity. Immunol Res. 2005; 31(1):47-55) (Kane L P, Weiss A. The PI-3 kinase/Akt pathway and T cell activation: pleiotropic pathways downstream of PIP3. Immunol. Rev. 2003; 192:7-20). Akt acts in a number of ways to orchestrate cell growth and to inhibit programmed cell death, e.g. through activation of NF-κB, stabilization of XIAP, sequestering of Bad, activation of mTOR and many other pathways. The PI3K/Akt pathway is recognized as a potential pharmaceutical target, and several drugs that are PI3K inhibitors have now entered clinical development.

From a further aspect the invention provides a method for identifying a compound suitable for the treatment of an autoimmune or inflammatory disease or transplant rejection comprising contacting TPP II with a compound to be screened, and identifying whether the compound inhibits the activity of TPP II.

Our data support the use of inhibitors of TPPII in the treatment of autoimmune or inflammatory diseases or transplant rejection.

Thus we have recognized that the PI3K/Akt pathway can be targeted for the purpose of down regulating the immune activation, thereby enabling therapy in auto-immune and inflammatory disease.

Over-activation of Akt in mice, as observed in Pten+/− mice and other transgenic strains, leads to rapid development of an autoimmune syndrome with over-production of auto-antibodies, and subsequent death of the mice (Di Cristofano, A, Kotsi, P, Peng, Y F, Cordon-Cardo, C, Elkon, K B, Pandolfi, P P. Impaired Fas response and autoimmunity in Pten+/− mice. Science. 1999; 285:2122-5). Also the selective over-expression of XIAP in transgenic mice leads to increased accumulation of T cells, although subtle compared what is observed in Pten-mutated mice (Conte, D, Liston, P, Wong, J W, Wright, K E, Korneluk, R G. Thymocyte-targeted overexpression of xiap transgene disrupts T lymphoid apoptosis and maturation. Proc Natl Acad Sci USA. 2001; 98:5049-54).

We have indicated a novel way to control Akt activation, and a class of compounds that are active in this process.

To test whether our TPPII inhibitors could improve the therapeutic effects of an anti-inflammatory drug in vivo, we examined growth of the T cell-derived lymphoma line EL-4 in vivo, in mice subjected to Cortisone-treatment. We used the derivate Dexamethasone at 5 mg/kg, a dose previously reported to cause thymocyte apoptosis, and a block of T cell responses in vivo (Brewer, J. A., Kanagawa, O., Sleckman, B. P., Muglia, L. J., Thymocyte apoptosis induced by T cell activation is mediated by glucocorticoids in vivo. J. Immunol. 2002, 169:1837-43.). The glucocorticoid receptor of T cells is crucial for curtailing lethal immune activation (Brewer J A, Khor B, Vogt S K, Muglia L M, Fujiwara H, Haegele K E, Sleckman B P, Muglia L J. T-cell glucocorticoid receptor is required to suppress COX-2-mediated lethal immune activation. Nat. Med. 2003 October; 9(10):1318-22.). Inhibited activation and proliferation of T cells in vivo by Dexamethasone-treatment is a standard method to treat patients with auto-immune, inflammatory as well as transplantation rejection diseases. It is however clear that disease symptoms, as well as immune activation and proliferation, are sometimes not controlled by Dexamethasone, or other Cortisone derivatives. Certain cytostatic drugs, e.g. Sendoxan or Cyclophosphamide, are treatment options when others have failed.

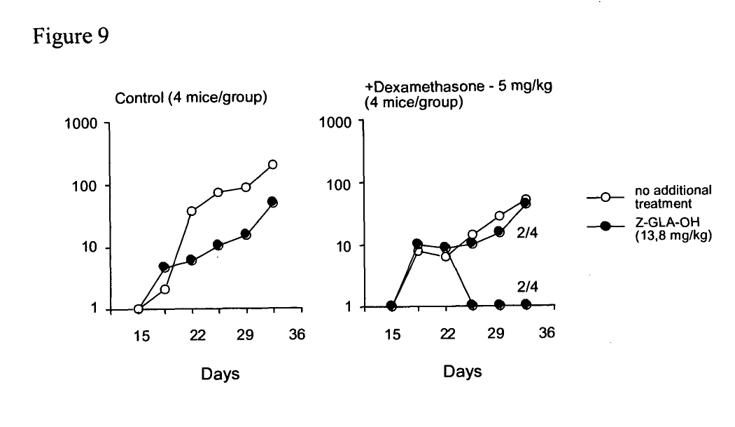

We inoculated 5×10⁶ EL-4 T-lymphoma cells into syngeneic C57BI/6 mice. These cells proliferate in vivo to form large tumors, and we observed some treatment effect of 5 mg/kg Dexamethasone twice weekly, i.e. reduced growth of EL-4 cells in vivo (FIG. 9). However, the addition of the TPPII inhibitor Z-GLA-OH increased the anti-proliferative effects of Dexamethasone in some of the mice. This illustrates that a TPPII inhibitor potentiates in vivo cell death of activated cells in mice treated with an anti-inflammatory drug. Thus, we thereby improve the action of Cortisone-derivates in the treatment of disease, which allows improved therapy in the diseases mentioned herein.

We have found that TPPII is a target for the treatment of auto-immune or inflammatory diseases. Inhibitors of TPPII may for example be used to treat patients with the following conditions, either in combination with other drugs (e.g. Dexamethazone, Sendoxan) or as monotherapy: 1. Systemic Lupus Erytematosus, 2. Rheumatoid Arthritis, 3. Multiple Sclerosis, 4. Sjögrens Syndrome, 5. Diabetes Mellitus Type I and II, 6. Psoriasis, 7. Eczema, 8. Ulcerous Colitis, 9. Chron's Disease or other auto-immune or inflammatory syndromes involving the immune system as cause of clinical disease symptoms.

In addition, mechanisms of inflammation and immune activation are important for disease pathogenesis in transplanted patients (e.g. Graft versus Host and Host versus Graft). The present invention provides compounds for use in the treatment of transplant rejection. For example, transplantation patients currently receive treatment with the mTOR inhibitor Rapamycin (and its analogues), and in one embodiment of the present invention treatment with inhibitors of TPPII is combined with and enhances such treatment. Nevertheless, the treatment with inhibitors of TPP II does not necessarily need to be in such combination therapy.

According to the present invention TPP II inhibitors are also useful in treating diseases or conditions that are a consequence of transplantation. For example, in transplanted patients a number of inflammation-related alterations can occur in the graft, e.g. vascular hypertrophy, and the treatment this or similar conditions is within the scope of the present invention.

Recent developments also support the theory that there is a strong inflammatory component, including the recruitment of T cells, in the pathogenesis of diseases where this was not previously recognized, such as in atherosclerosis. The possibility of applying TPPII inhibitors to relieve immune activation to inhibit inflammation, auto-immune and transplantation rejection pathogenesis allows improved therapy in diseases where such states are observed.

TPP II accepts a relatively broad range of substrates. All the compounds falling within formula (i) are peptides or peptide analogues. Compounds of formulae (i) are readily synthesizable by methods known in the art (see for example Ganellin et al., J. Med. Chem. 2000, 43, 664-674) or are readily commercially available (for example from Bachem AG). In a preferred aspect the compound may be selected from formulae (i). Such tripeptides and derivatives are particularly effective therapeutic agents.

According to the invention the compound for use in the treatment of autoimmune or inflammatory diseases or transplant rejection may be a compound which is known to be a TPP II inhibitor in vivo.

For example, the compound may be selected from compounds identified in Winter et al., Journal of Molecular Graphics and Modelling 2005, 23, 409-418 as TPP II inhibitors. The compounds may be selected from the following formula (ii) because these compounds are particularly suited to the TPP II pharmacophore:

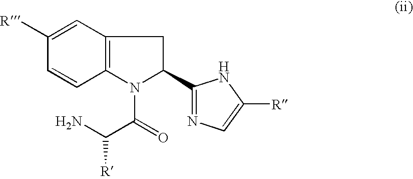

wherein R′ is H, CH₃, CH₂CH₃, CH₂CH₂CH₃ or CH(CH₃)₂,
R″ is H, CH₂CH₃, CH₂CH₂CH₃, CH(CH₃)₂, CH₂CH₂CH₂CH₃, CH₂CH(CH₃)₂, CH(CH₃)CH₂CH₃ or C(CH₃)₃, and
R′″ is H, CH₃, OCH₃, F, Cl or Br;

Compounds of formula (ii) are synthesizable by known methods (see for example Winter et al., Journal of Molecular Graphics and Modelling 2005, 23, 409-418 and Breslin et at., Bioorg. Med. Chem. Lett. 2003, 13, 4467-4471).

Also by way of example, the compound may be selected from compounds identified in U.S. Pat. No. 6,335,360 of Schwartz et al. as TPP II inhibitors. Such compounds include those of the following formula (iii).

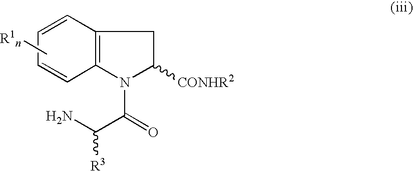

wherein:
each R¹ may be the same or different, and is selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C₁-C₆) alkynyl, optionally substituted by one or more radicals selected from the group consisting of halogen and OH, X(C₁-C₆)alkyl, wherein X is S, 0 or OCO, and the alkyl is optionally substituted by one or more radicals selected from the group consisting of halogen and OH; SO₂ (C₁-C₆)alkyl, optionally substituted by at least one halogen, YSO₃H, YSO₂ (C₁-C₆)alkyl, wherein Y is O or NH and the alkyl is optionally substituted by at least one halogen, a diradical —X1-(C₁-C₂)alkylene-X1-wherein X₁ is O or S; and a benzene ring fused to the indoline ring;
n is from 0 to 4;
R² is CH₂R⁴, wherein R⁴ is C₁-C₆ alkyl substituted by one or more radicals selected from the group consisting of halogen and OH; (CH₂)ₚZ(CH₂)ₚCH₃, wherein Z is O or S, p is from 0 to 5 and q is from 0 to 5, provided that p+q is from 0 to 5; (C₂-C₆) unsaturated alkyl; or (C₃-C₆) cycloalkyl;
or R² is (C₁-C₆)alkyl or O(C₁-C₆)alkyl, each optionally substituted by at least one halogen;
R³ is H; (C₁-C₆)alkyl optionally substituted by at least one halogen; (CH₂)ₚZR⁵ wherein p is from 1 to 3, Z is O or S and R⁵ is H or (C₁-C₃)alkyl; benzyl.

Compounds of formula (iii) are readily synthesizable by known methods (see for example U.S. Pat. No. 6,335,360 of Schwartz et al.).

Nevertheless, it is preferred that the compound be selected from formulae (i) and (ii), more preferably formula (i).

It is also possible for the compound to be a compound of formula (i) wherein R^{N1}, R^{N2} and R^{C1} are as defined above or in any of the preferences below and wherein:
A¹ is G, A, V, L, I, P, S, T, C, N, Q, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine,
A² is G, A, V, L, I, P, S, T, C, N, Q, F, Y, W, K, R, histidine, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine, alpha,gamma-diaminobutyric acid or 4,5-dehydro-lysine, and
A³ is G, A, V, L, I, P, S, T, C, N, Q, D, E, F, Y, W, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine.

### Preferred Compounds of Formula (i)

Various groups and specific examples of compounds of formula (i) are preferred.

In general, amino acids of natural (L) configuration are preferred, particularly at the A² position.

In general, it is preferred that R^{N1} is hydrogen, and that
R^{N2} is:
   R^{N3},
   (linker1)-R^{N3},
   CO-(linker1)R^{N3}, or
   CO—O-(linked)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl;
   benzyl;
   phenyl; or
   monocyclic heteroaryl.
In general, it is preferred that R^{C1} is:
   O—R^{C2},
   O-(linker2)-R^{C2}, or
   NH-(linker2)R^{C2}
wherein
(linker1) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C_{—6} alkyl;
   benzyl;
   phenyl; or
   monocyclic C₁₋₁₀ heteroaryl.

In general, with regard to the substituents at the N-terminus, it is further preferred that:

R^{N1} is hydrogen, and
R^{N2} is hydrogen, C(═O)—O-(linker1)-R^{N3} or C(═O)-(linker1)-R^{N3},
(linker1) is CH₂ or CH═CH, and
R^{N3} is phenyl or 2-furyl.

It is further preferred that

R^{N1} is hydrogen,
R^{N2} is hydrogen, C(═O)—OCH₂Ph or C(═O)—CH═CH-(2-furyl).

Another preferred grouping for the substituents on the N-terminus is such that:

R^{N1} is hydrogen, and
R^{N2} is a is benzyloxycarbonyl, benzyl, benzoyl, tert-butyloxycarbonyl, 9-fluorenylmethoxycarbonyl or FA, more preferably benzyloxycarbonyl or FA.

In general, with regard to the substituents at the C-terminus, it is preferred that:

R^{C1} is OH, O—C₁₋₆ alkyl, O-C₁₋₆ alkyl-phenyl, NH—C₁₋₆ alkyl, or NH—C₁₋₆ alkyl-phenyl, more preferably OH.

Several preferred groups are as follows.

Group (i)(a):

A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

Group (i)(b):

A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

Group (i)(c):

A¹ is G, A or 2-aminobutyric acid,
A² is L, I, norleucine, V, norvaline, tert-butyl alanine, 4,5-dehydro-leucine, alto-isoleucine or 2-allylglycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O-C₁₋₆ alkyl, O—C₁₋₆ alkyl-phenyl, NH—C₁₋₆ alkyl, or NH—C₁₋₆ alkyl-phenyl.

Group (i)(d):

### A¹ is G or A,

A² is L, I, or norleucine,

### A³ is G or A,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

A first set of specific preferred compounds are those in which:

### A¹ is G,

### A² is L,

A³ is G, A, V, L, I, P, F, W, D, E, Y, 2-aminobutyric acid, norvaline or tert-butyl glycine, more preferably G, A, V, P, 2-aminobutyric acid or norvaline, more preferably G or A,
R^{N1} is hydrogen,
R^{N2} is benzyloxycarbonyl, and

### R^{C1} is OH.

A second set of specific preferred compounds are those in which:

### A¹ is G,

A² is G, A, V, L, I, P, F, W, C, S, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine, more preferably L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine, more preferably L, I, norleucine, V, norvaline, tert-butyl alanine, 4,5-dehydro-leucine, allo-isoleucine or 2-allylglycine, more preferably L, I, or norleucine,

### A³ is A,

R^{N1} is hydrogen,
R^{N2} is benzyloxycarbonyl, and

### R^{C1} is OH.

A third set of specific preferred compounds are those in which:

A¹ is G, A, V, L, I, P, 2-aminobutyric acid, norvaline or tert-butyl glycine, more preferably G,
A or 2-aminobutyric acid, more preferably G or A,

### A² is L,

### A³ is A,

R^{N1} is hydrogen,
R^{N2} is benzyloxycarbonyl, and

### R^{C1} is OH.

Preferably the sequence A¹-A²-A³ is GLA, GLF, GVA, GIA, GPA or ALA, most preferably GLA, and:

R^{N1} is hydrogen,
R^{N2} is benzyloxycarbonyl, and

### R^{C1} is OH.

Where alkyl groups are described as saturated or unsaturated, this encompasses alkyl, alkenyl and alkynyl hydrocarbon moieties.

C₁₋₆ alkyl is preferably C₁₋₄ alkyl, more preferably methyl, ethyl, n-propyl, isopropyl, or butyl (branched or unbranched), most preferably methyl.

C₃₋₁₂ cycloalkyl is preferably C₅₋₁₀ cycloalkyl, more preferably C₅₋₇ cycloalkyl.

“aryl” is an aromatic group, preferably phenyl or naphthyl,

“hetero” as part of a word means containing one or more heteroatom(s) preferably selected from N, O and S.

“heteroaryl” is preferably pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzimidazolyl, benzthiazolyl, purinyl, cinnolinyl or pteridinyl.

“non-aromatic heterocyclyl” is preferably pyrrolidinyl, piperidyl, piperazinyl, morpholinyl, tetrahydrofuranyl or monosaccharide.

“halogen” is preferably Cl or F, more preferably Cl.

### Further Preferred Compounds of Formula (i)

In general, A¹ may preferably be selected from G, A or 2-aminobutyric acid; more preferably G or A, most preferably G.

In general, A² may preferably be selected from L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine or tert-butyl glycine; more preferably L, I, norleucine, V, norvaline, tert-butyl alanine, 4,5-dehydro-leucine, allo-isoleucine, 2-allylglycine, P or K; more preferably L, I, norleucine, P or K; more preferably L or P.

In general, A³ may preferably be selected from G, A, V, P, 2-aminobutyric acid or norvaline; more preferably G or A. One general preference is that A³ is G. Another general preference is that A³ is A, particularly when R^{C1} is OH.

In general, it is preferred that R^{N1} is hydrogen.

In general, R^{N2} is preferably:
R^{N3},
(linker1)-R^{N3},
CO-(linker1)-R^{N3}, or

CO—O-(linker1)-R³,
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl;
   benzyl;
   phenyl; or
   monocyclic heteroaryl.

In general, R^{N2} is more preferably hydrogen, benzyloxycarbonyl, benzyl, benzoyl, tert-butyloxycarbonyl, 9-fluorenylmethoxycarbonyl or FA, more preferably hydrogen, benzyloxycarbonyl or FA.

In general, it is preferred that R^{C1} is:
O—R^{C2},
O-(linker2)—R^{C2}, or
NH-(linker2)R^{C2}
wherein
(linker2) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C₁₋₅ alkyl;
   benzyl;
   phenyl; or
   monocyclic C₁₋₁₀ heteroaryl.

In general, R^{C1} is more preferably OH, O—C₁₋₆ alkyl, O—C₁ alkyl-phenyl, NH₂, NH—C₁₋₆ alkyl, or NH—C₁₋₆ alkyl-phenyl, more preferably OH, O—C₁₋₆ alkyl, NH₂, or NH—C₁₋₆ alkyl, more preferably OH or NH₂.

Compounds of particular interest include those wherein A² is P.

Compounds of particular interest include those wherein R^{C1} is NH₂.

In general the following amino acids are less preferred for A³: F, W, D, E and Y. Similarly, in general A³ may be selected not to be P and/or E due to compounds containing these exhibiting lower activity.

### Preferred Compounds of Formula (ii)

Compounds of formula (ii) are preferably such that:

### R′ is CH₂CH₃ or CH₂CH₂CH₃,

### R″ is CH₂CH₂CH₃ or CH(CH₃)₂, and

### R′″ is H or Cl.

Preferred Compounds of Formula (iii)

Various preferred groups and specific examples of compounds of formula (iii) are as defined in any of the claims, taken separately, of U.S. Pat. No. 6,335,360 B1 of Schwartz et al.

One example of a therapeutic compound of formula (i) is Z-GLA-OH, i.e. tripeptide GLA which is derivatized at the N-terminus with a Z group and which is not derivatized at the C-terminus. Z denotes benzyloxycarbonyl. This is a compound of formula (i) wherein R^{N1} is H, R^{N2} is Z, A¹ is G, A² is L, A³ is A and R^{C1} is OH. This compound is available commercially from Bachem AG and has been found to inhibit the bacterial homologue of the eukaryotic TPP II, Subtilisin. Z-GLA-OH is of low cost and works well experimentally.

Whilst preferred compounds include those containing GLA, such as Z-GLA-OH, Bn-GLA-OH, FA-GLA-OH and H-GLA-OH, for example Z-GLA-OH; according to the present invention any disclosures of any compounds or groups of compounds herein may optionally be subject to the proviso that the sequence A¹A²A³ is not GLA, or the proviso that the compound is not selected from the group consisting of Z-GLA-OH, Bn-GLA-OH, FA-GLA-OH or H-GLA-OH, or the proviso that the compound is not Z-GLA-OH.

In the treatment of autoimmune or inflammatory diseases or transplant rejection Z-GLA-OH or other compounds described herein may be administered.

Other preferred compounds include those wherein A¹A²A³ is GPG, such as GPG-NH₂ or Z-GPG-NH₂.

The skilled person will be aware that the compounds described herein may be administered in any suitable manner. For example, the administration may be parenteral, such as intravenous or subcutaneous, oral, transdermal, intranasal, by inhalation, or rectal. In one preferred embodiment the compounds are administered by injection.

Examples of pharmaceutically acceptable addition salts for use in the pharmaceutical compositions of the present invention include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids. The pharmaceutically acceptable excipients described herein, for example, vehicles, adjuvants, carriers or diluents, are well-known to those who are skilled in the art and are readily available to the public. The pharmaceutically acceptable carrier may be one that is chemically inert to the active compounds and that has no detrimental side effects or toxicity under the conditions of use. Pharmaceutical formulations are found e.g. in Remington: The Science and Practice of Pharmacy, 19th ed., Mack Printing Company, Easton, Pa. (1995).

The composition may be prepared for any route of administration, e.g. oral, intravenous, cutaneous or subcutaneous, nasal, intramuscular, or intraperitoneal. The precise nature of the carrier or other material will depend on the route of administration. For a parenteral administration, a parenterally acceptable aqueous solution is employed, which is pyrogen free and has requisite pH, tonicity and stability. Those skilled in the art are well able to prepare suitable solutions and numerous methods are described in the literature. A brief review of methods of drug delivery is also found in e.g. Langer, Science 249:1527-1533 (1990).

The dose administered to a mammal, particularly a human, in the context of the present invention should be sufficient to effect a therapeutic response in the mammal over a reasonable time frame. One skilled in the art will recognize that dosage will depend upon a variety of factors including the age, condition and body weight of the patient, as well as the stage/severity of the disease. The dose will also be determined by the route (administration form) timing and frequency of administration. In the case of oral administration the dosage can vary for example from about 0.01 mg to about 10 g, preferably from about 1 mg to about 8 g, preferably from about 10 mg to about 5 g, more preferably from about 10 mg to about 2 g, more preferably from about 100 mg to about 1 g per day of a compound or the corresponding amount of a pharmaceutically acceptable salt thereof.

Treatment may be applied in a single dose, or periodically as a course of treatment.

It is clear to the skilled person how to screen compounds for their inhibition of the activity of TPP II. TPP II protein may be purified in a first step, and a TPP II-preferred fluorogenic substrate may be used in a second step. This results in an effective method to measure TPP II activity.

It is not necessary to achieve a particularly high level of purification, and conventional simple techniques can be used to obtain TPP II of sufficient quality to use in a screening method. In one non-limiting example of purification of TPP II, 100×10⁶ cells (such as EL-4 cells) were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

It is possible to test the activity of TPP II using for example the substrate AAF-AMC (Sigma, St. Louis, Mo.). This may for example be used at 100 uM concentration in 100 ul of test buffer composed of 50 mM Tri Base pH 7.5, 5 mM MgCl₂ and 1 mM DTT. It is possible to stop reactions using dilution with 900 ul 1% SDS solution. Cleavage activity may be measured for example by emission at 460 nm in a LS50B Luminescence Spectrometer (Perkin Elmer, Boston, Mass.).

The compounds of use in the present invention may be defined as those which result in partial or preferably complete treatment of autoimmune or inflammatory diseases or transplant rejection in vivo.

The compounds used in the present invention are sufficiently serum-stable, i.e. in vivo they retain their identity long enough to exert the desired therapeutic effect.

The present invention is described in more detail in the non-limiting Examples below with reference to the accompanying drawings which are now summarised.

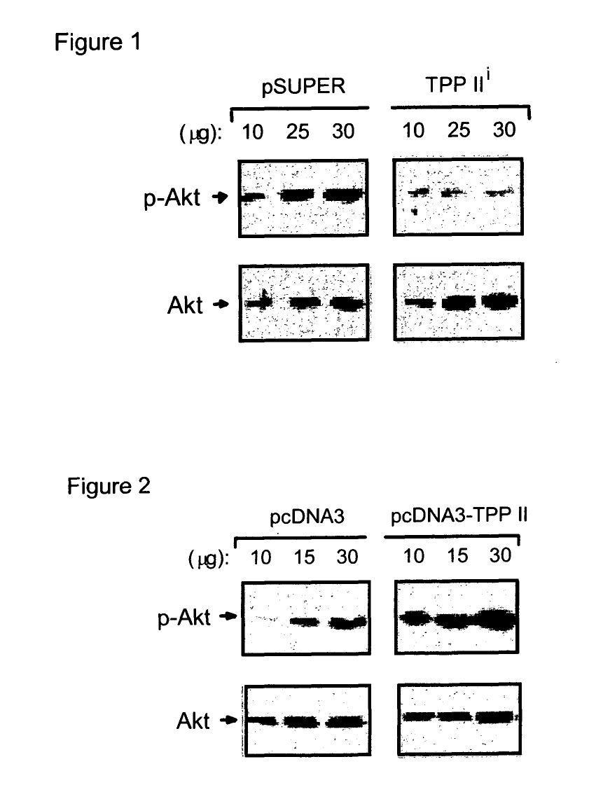

FIG. 1 shows a Western blotting analysis of Akt kinase expression, total Akt and Ser473-phosphorylated (p-Akt), in EL-4.wt control versus EL-4.TPPIIⁱ cells (“micro-g” denotes the amount of cellular lysate loaded for Western blotting);

FIG. 2 shows a Western blotting analysis of Akt kinase expression, total Akt and Ser473-phosphorylated (p-Akt), in EL-4.pcDNA3 versus EL-4.pcDNA3-TPPII cells (“micro-g” denotes the amount of cellular lysate loaded for Western blotting);

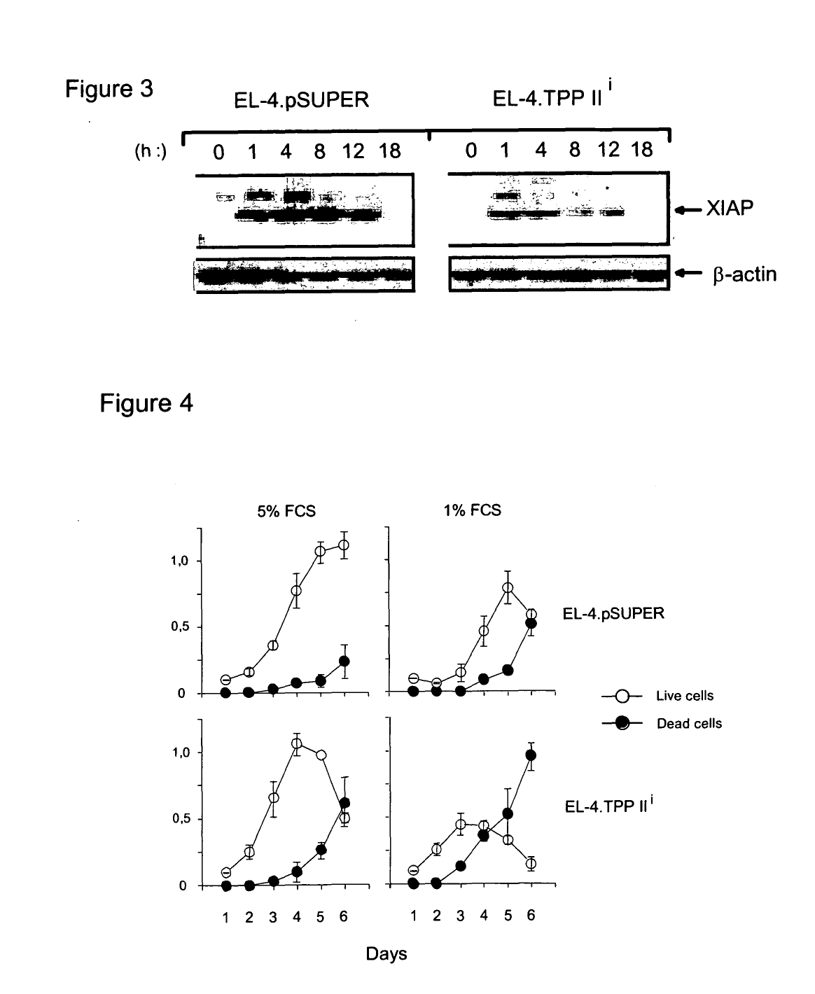

FIG. 3 shows expression of XIAP as analyzed by Western blotting, following treatment with 25 micro-M Etoposide;

FIG. 4 shows growth in vitro of EL-4.wt and EL-4.TPPIIⁱ cells in cell culture medium with either high (5%, left) or low (1%, right) serum content [both live (empty circles) and dead (filled circles) cells were counted];

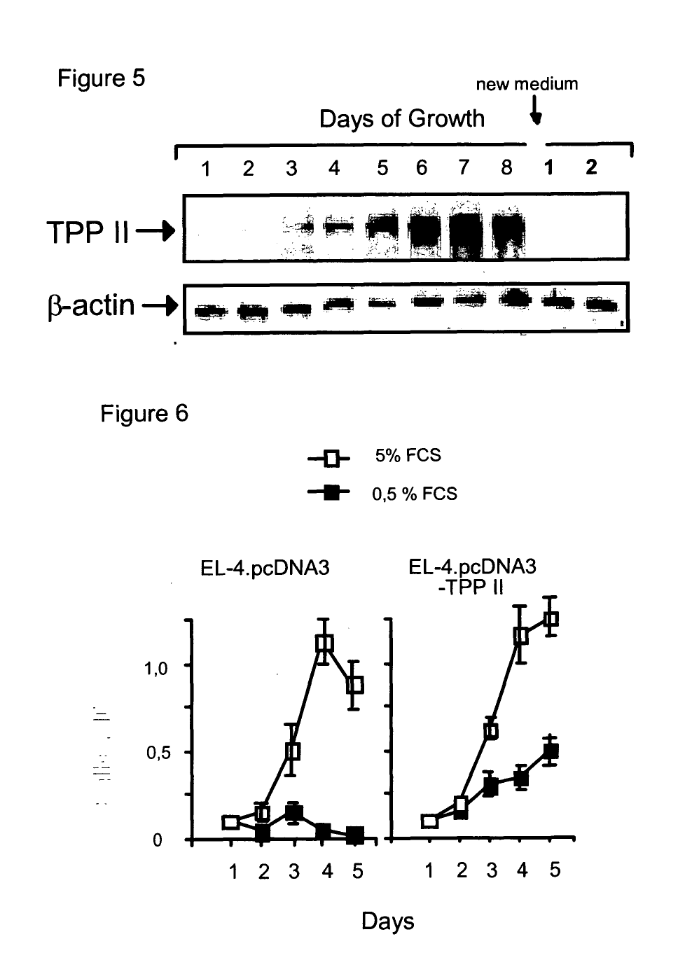

FIG. 5 shows TPPII expression in EL-4.wt cells seeded at 100 000/ml at day 1 without replenishment of medium, until day 8 (indicated by arrow);

FIG. 6 shows growth in vitro of EL-4.pcDNA3 and EL-4.pcDNA3-TPPII cells in cell culture medium with either high (5%, empty circles) or low (0,5%, filled circles) serum content;

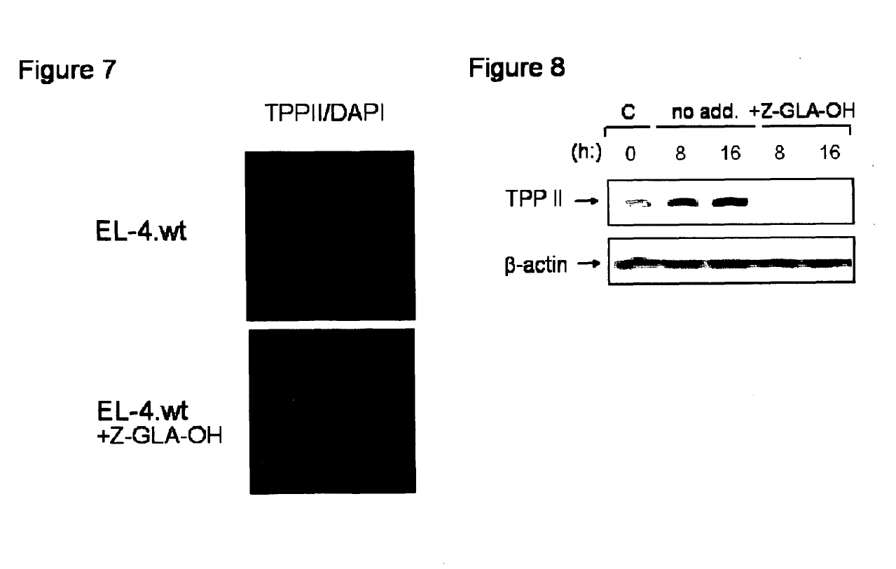

FIG. 7 shows immuno-cytochemical staining to test whether targeting of TPPII in live cells affected enzyme expression or distribution;

FIG. 8 shows, by means of a Western blotting analysis, the targeting and depletion of TPPII in live cells by treatment with TPPII inhibitors;

FIG. 9 shows growth of EL-4 T-lymphoma cells in vivo, in syngeneic mice, treated with Dexamethasone (5 mg/kg) and/or Z-GLA-OH (13.8 mg/kg), or left untreated.

### EXAMPLES

The materials and methods used were as follows.

Cells and Culture Conditions. EL-4 is a Benzpyrene-induced lymphoma cell line derived from the C57BI/6 mouse strain. EL-4.wt and EL-4.TPPIIⁱ are EL-4 cells transfected with the pSUPER vector (Brummelkamp, T R, Bernards, R, Agami, R. A system for stable expression of short interfering RNAs in mammalian cells. Science 2002; 296:550-3), empty versus containing the siRNA directed against TPPII. For generation of stable transfectants, 5×10⁶ cells were washed in PBS, then resuspended into 500 micro-I of PBS in a Bio-Rad gene-pulser and pulsed with 10 micro-g DNA and 250 V at 960 micro-F; and selected by resistance to G418.

Enzyme Inhibitors. NLVS is an inhibitor of the proteasome that preferentially targets the chymotryptic peptidase activity, and efficiently inhibits proteasomal degradation in live cells. Butabindide is described in the literature (Rose, C, Vargas, F, Facchinetti, P, Bourgeat, P, Bambal, R B, Bishop, P B, et. al. Characterization and inhibition of a cholecystokinin-inactivating serine peptidase. Nature 1996; 380:403-9). Z-Gly-Leu-Ala-OH (Z-GLA-OH) is an inhibitor of Subtilisin (Bachem, Weil am Rhein, Germany), a bacterial enzyme with an active site that is homologous to that of TPPII. Wortmannin is an inhibitor of PIKK (PI3-kinase-related)-family kinases (Sigma, St. Louis, Mo.). All inhibitors were dissolved in DMSO and stored at −20° C. until use.

Protein Purification, Peptidase Assays and Analysis of DNA Fragmentation. 100×10⁶ cells were sedimented and lysed by vortexing in glass beads and homogenisation buffer (50 mM Tris Base pH 7.5, 250 mM Sucrose, 5 mM MgCl₂, 1 mM DTT). Cellular lysates were submitted to differential centrifugation where a supernatant from a 1 hour centrifugation at 100,000×g (cytosol) was submitted to 100,000×g centrifugation for 3-5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 micro-g of high molecular weight protein was used as enzyme in peptidase assays or in Western blotting for TPP II expression. To test the activity of TPP II we used the substrate AAF-AMC (Sigma, St. Louis, Mo.), at 100 micro-M concentration in 100 micro-I of test buffer composed of 50 mM Tri Base pH 7.5, 5 mM MgCl₂ and 1 mM DTT. Cleavage activity was measured by emission at 460 nm in a LS50B Luminescence Spectrometer (Perkin Elmer, Boston, Mass.). For analysis of DNA fragmentation cells were seeded in 12-well plates at 10⁶ cells/ml and exposed to 25 micro-M etoposide, a DNA topoisomerase II inhibitor commonly used as an apoptosis-inducing agent, to starvation (50% PBS). Cells were seeded at 10⁶ cells/ml in 12-well plates and incubated for the indicated times, usually 18-24 hours. DNA from EL-4 control and adapted cells was purified by standard chloroform extraction, and 2.5 micro-g of DNA was loaded on 1.8% agarose gel for detection of DNA from apoptotic cells.

Antibodies and Antisera. The following molecules were detected by the antibodies specified: GFP by rabbit anti-GFP serum (Molecular Probes Europe, Breda, The Netherlands); 19S proteasomal complexes by anti-Rpt6 (19S base ATPase subunit), 20S proteasomal complexes by (Affinity, Exeter, UK); For detection of TPPII we used chicken anti-TPPII serum (Immunsystem, Uppsala, Sweden). In experiments where whole cell lysates were used for western blotting of TPPII, i.e. fractions not enriched for TPPII, TPPII fell below the limit of detection in cells not exposed to stress. Western blotting was performed by standard techniques. Protein concentration was measured by BCA Protein Assay Reagent (Pierce Chemical Co.). 5 micro-g of protein was loaded per lane for separation by SDS/PAGE unless stated otherwise.

Immunocytochemistry. Cells were attached to glass cover slips through cytospin and fixed in acetone:methanol (1:1) for 1 hour; then the slides were rehydrated in BSS buffer for 1 hour. The first antibody was added and remained for 1 hour until a brief wash in BSS, after which a secondary conjugate (anti-rabbit-FITC) was added and incubated for 1 hour.

Then the slides were washed and stained with Hoescht 333258 for 30 min. Finally, the slides were mounted with DABCO mounting buffer and kept at 4° C. until analysis.

Abbreviations list: NLVS, 4-hydroxy-5-iodo-3-nitrophenylacetyl-Leu-Leu-Leu-vinyl sulphone; PIKKs, Phosphoinositide-3-OH-kinase-related kinases; TPPII, Tripeptidyl-peptidase II; FA=3-(2-furyl)acryloyl.

Standard abbreviations are used for chemicals and amino acids herein.

| | | Alternative |
|---|---|---|
| Abbreviation | | abbreviation |
| A | Alanine | Ala |
| R | Arginine | Arg |
| N | Asparagine | Asn |
| D | Aspartic acid | Asp |
| C | Cysteine | Cys |
| E | Glutamic Acid | Glu |
| Q | Glutamine | Gln |
| G | Glycine | Gly |
| H | Histidine | His |
| I | Isoleucine | Ile |
| L | Leucine | Leu |
| K | Lysine | Lys |
| M | Methionine | Met |
| F | Phenylalanine | Phe |
| P | Proline | Pro |
| S | Serine | Ser |
| T | Threonine | Thr |
| W | Tryptophan | Trp |
| Y | Tyrosine | Tyr |
| V | Valine | Val |

The invention also makes use of several unnatural alpha-amino acids.

| Abbrevia- | | |
|---|---|---|
| tion | | SIDE CHAIN |
| Abu | 2-aminobutyric acid | CH₂CH₃ |
| Nva | norvaline | CH₂CH₂CH₃ |
| Nle | norleucine | CH₂CH₂CH₂CH₃ |
| | tert-butyl alanine | CH₂C(CH₃)₃ |
| | alpha-methyl leucine | (CH₃)(CH₂C(CH₃)CH₃) |
| | 4,5-dehydro-leucine | CH₂C(═CH₂)CH₃ |
| | allo-isoleucine | CH(CH₃)CH₂CH₃ |
| | alpha-methyl valine | (CH₃)CH(CH₃)(CH₃) |
| | tert-butyl glycine | C(CH₃)₃ |
| | 2-allylglycine | CH₂CH═CH₂ |
| Orn | Ornithine | CH₂CH₂CH₂NH₂ |
| Dab | alpha,gamma-diaminobutyric acid | CH₂CH₂NH₂ |
| | 4,5-dehydro-lysine | CH₂CH═CHCH₂NH₂ |

The Figures show that TPPII controls pathways which respond to nutritional status; in particular TPPII controls Akt activation, growth factor requirements and cell survival.

### Example 1

With reference to the Figures, we tested whether TPPII regulated Akt activation, by Western blot analysis of Akt kinase, total Akt protein as well as Phospho-Ser473-Akt. We used several variants of the T cell-derived lymphoma cell line EL-4, EL-4.wt with normal TPPII expression (transfected with empty pSUPER vector—Brummelkamp TR, Bernards R, Agami R. A system for stable expression of short interfering RNAs in mammalian cells. Science 2002; 296:550-3); EL-4.TPPIIⁱ with inhibited TPPII expression (transfected with a pSUPER vector encoding an siRNA directed towards TPPII); EL-4.pcDNA3 with normal TPPII expression (transfected with empty pcDNA3-neo vector); EL-4.pcDNA3-TPPII (transfected with a TPPII encoding plasmid). All these cell lines were stable transfectants. We detected substantial levels of Phospho-Ser473-Akt in lysates of EL-4.wt cells, i.e. indicating an activated state of Akt kinase (FIG. 1). However, EL-4.TPPIIi cells displayed very low levels of Ser473-Akt, suggesting reduced activation of this kinase (FIG. 1).

### Example 2

In addition, we find increased Ser473 phosphorylation of Akt in EL-4.pcDNA3-TPPII, in comparison to EL-4.pcDNA3 control cells further supporting that TPPII expression controls Akt-Ser473 phosphorylation (FIG. 2).

### Example 3

One consequence of Akt activation is increased stability of XIAP (an endogenous caspase inhibitor), a downstream target of Akt [Dan H C, Sun M, Kaneko S, Feldman R I, Nicosia S V, Wang H G, et. al. Akt phosphorylation and stabilization of X-linked inhibitor of apoptosis protein (XIAP). J Biol Chem 2004; 279:5405-12)]. We treated EL-4.wt and EL-4.TPPIIi cells with etoposide and the expression of XIAP was analyzed by western blotting of cellular lysates up to 18 hours. We find that degradation of XIAP was substantially slower in EL-4.wt compared to EL-4.TPP IIⁱ cells (FIG. 3).

These data support the theory that TPP II expression regulates signaling by Akt kinase as well as XIAP expression, a downstream target.

### Example 4

The status of Akt activation was in line with the serum requirements during in vitro cell growth of EL-4.wt versus EL-4.TPPIIⁱ cells. In normal medium (5% serum) EL-4.TPPIIⁱ cells showed an increased rate of proliferation, compared to EL-4.wt, but also an increased accumulation of dead cells (FIG. 4). Further, by lowering serum concentrations to 1% this accumulation was accelerated, compared to EL-4.wt cells (FIG. 4).

### Example 5

In addition, TPPII was strongly induced in EL-4.wt days 5-7 (following seeding of cells at 100 000/ml), supporting the theory that TPPII was important for cells approaching starvation (FIG. 5). Replenishment of medium down regulated TPPII expression (FIG. 5, arrow).

### Example 6

In addition, EL-4.pcDNA3-TPPII cells were able perform limited growth in 0.5% serum, which EL-4.pcDNA3 cells did not (FIG. 6). These results indicated that TPPII expression was important for Akt Ser473 phosphorylation and the requirements of growth factors of a T cell derived lymphoma cell line.

### Examples 7 and 8

In support of the theory that TPPII inhibitors control the expression of TPPII in live cells, we refer inter alia to FIGS. 7 and 8 herein. The present invention utilizes a class of TPPII inhibitors which are suitable for in vivo treatment. These include the tri-peptide inhibitor Z-GLA-OH. We performed immuno-cytochemical staining to test whether targeting of TPPII in live cells affected enzyme expression or distribution, by the use of a chicken anti-TPPII serum. In untreated EL-4 lymphoma cells we found that TPPII was distributed in predominantly the cytosol, but also with some nuclear staining. By the incubation of EL-4 cells for 2 hours with 10 micro-M of Z-GLA-OH we found a rapid decrease in the detection of TPPII, to almost undetectable levels (FIG. 7, bottom); similar results were obtained by incubation with the TPPII inhibitor butabindide. Further, by western blotting analysis we also found a rapid clearance of TPPII protein, giving further support that TPPII is targeted and also depleted by treatment with TPPII inhibitors (FIG. 8).

### Example 9

### TPPII Inhibitors Potentiate In Vivo Cell Death of Activated Cells in Mice Treated with an Anti-Inflammatory Drug

5×10⁶ EL-4 T-lymphoma cells were inoculated subcutaneously into syngeneic C57BI/6 mice. Once tumours were established these were left untreated (“Control”) treated with 5 mg/kg of Dexamethasone alone or in combination with 13.8 mg/kg of Z-GLA-OH (twice weekly), or with Z-GLA-OH alone. The size of EL-4 lymphoma tumours were measured manually twice weekly. The vertical scale in FIG. 9 indicates size in mm³.

### Example 10

### In Vitro Testing of Di- and Tri-Peptides and Derivatives

Table 1 contains in vitro data, in fluorometric units which are arbitrary but relative, for the inhibition of cleavage of AAF-AMC (H-Ala-Ala-7-amido-4-methylcoumarin) by compounds at several concentrations. Some beneficial effect is seen for most of the compounds tested.

TPP II protein was enriched, and then a TPP II-preferred fluorogenic substrate AAF-AMC was used. 100×10⁶ cells were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

To test the activity of TPP II we used the substrate and AAF-AMC (Sigma, St. Louis, Mo.), at 100 uM concentration in 100 ul of test buffer composed of 50 mM Tri Base pH 7.5, 5 mM MgCl₂ and 1 mM DTT. To stop reactions we used dilution with 900 ul 1% SDS solution. Cleavage activity was measured by emission at 460 nm in a LS50B Luminescence Spectrometer (Perkin Elmer, Boston, Mass.).

FA=3-(2-furyl)acryloyl; PBS=phosphate-buffered saline. The text (Z, FA, H, etc.) at the start of each compound name is the substituent at the N-terminus; H indicates that the N-terminus is free NH₂. The text (OH, NBu, etc.) at the end of each compound name is the substituent at the C-terminus; OH indicates that the C-terminus is free CO₂H.

TABLE 1

| | 100 | | | | | | |
|---|---|---|---|---|---|---|---|
| Compound | uM | 10 uM | 1 uM | 100 nM | 10 nM | 1 nM | 0 |
| Z-GL-OH | 23.14 | 23.60 | 24.18 | 34.6 | 34.07 | 44.53 | 49.55 |
| (comparative) | 24.99 | 24.72 | 24.4 | 33.02 | 33.85 | 44.21 | 49.82 |
| | 23.69 | 24.59 | 24.29 | 34.6 | 34.38 | 43.62 | 49.51 |
| mean | 23.94 | 24.30 | 24.29 | 34.07 | 34.1 | 44.12 | 49.63 |
| Z-GLG-OH | 14.44 | 17.49 | 23.79 | 31.49 | 34.4 | 43.42 | 48.58 |
| | 15.02 | 17.58 | 24.85 | 28.64 | 34.16 | 44.02 | 49.03 |
| | 15.8 | 17.44 | 24.63 | 26.13 | 34.27 | 43.73 | 49.2 |
| mean | 15.09 | 17.50 | 24.42 | 28.75 | 34.28 | 43.72 | 48.94 |
| Z-GGA-OH | 15.5 | 16.65 | 21.37 | 24.27 | 36.01 | 43.42 | 51.19 |
| | 15.27 | 17.27 | 22.14 | 31.54 | 36.59 | 43.87 | 48.44 |
| | 15.78 | 17.18 | 22.62 | 31.61 | 36.73 | 44.14 | 48.48 |
| mean | 15.52 | 17.03 | 22.04 | 29.14 | 36.44 | 43.81 | 49.37 |
| FA-GLA-OH | 6.34 | 14.35 | 19.99 | 23.33 | 31.19 | 43.18 | 49.96 |
| | 4.05 | 8.14 | 16.21 | 23.87 | 33.88 | 43.49 | 48.4 |
| | 4.69 | 9.44 | 14.78 | 24.09 | 33.9 | 43.68 | 49.43 |
| mean | 5.03 | 10.64 | 16.99 | 23.76 | 32.99 | 43.45 | 49.26 |
| H-APA-OH | 13.55 | 14.35 | 23.94 | 24.26 | 28.85 | 44.05 | 48.84 |
| | 8.46 | 14.64 | 24.49 | 24.48 | 29.39 | 41.76 | 49.32 |
| | 7.65 | 14.91 | 25.04 | 28.44 | 29.44 | 43.84 | 49.16 |
| mean | 9.89 | 14.63 | 24.49 | 25.73 | 29.23 | 43.22 | 49.11 |
| H-GLA-OH | 8.37 | 12.4 | 15.53 | 17.58 | 22.67 | 36.63 | 48.16 |
| | 7.42 | 12.53 | 19.03 | 17.94 | 23.33 | 38.42 | 49.91 |
| | 7.12 | 14.66 | 18.34 | 17.53 | 22.93 | 39.4 | 48.18 |
| mean | 7.64 | 13.20 | 17.63 | 17.68 | 22.98 | 38.15 | 48.75 |
| Bn-GLA-OH | 12.92 | 17.74 | 21.14 | 23.01 | 33.30 | 43.67 | 48.53 |
| | 11.17 | 14.86 | 21.54 | 22.71 | 33.45 | 42.91 | 47.02 |
| | 9.65 | 13.38 | 22.01 | 22.90 | 33.40 | 41.17 | 49.55 |
| mean | 11.25 | 15.33 | 21.56 | 22.87 | 33.38 | 42.58 | 48.37 |
| Z-GKA-OH | 8.17 | 12.48 | 14.49 | 21.62 | 23.57 | 42.13 | 49.82 |
| | 9.44 | 14.52 | 16.43 | 21.98 | 23.95 | 42.02 | 49 |
| | 9.44 | 14.82 | 15.03 | 21.52 | 24.36 | 42.51 | 47.7 |
| mean | 9.02 | 13.94 | 15.32 | 21.71 | 23.96 | 42.22 | 48.84 |
| Z-GLA-Nbu | 11.16 | 13.06 | 23.89 | 32.24 | 34.06 | 38.14 | 47.34 |
| | 13.86 | 14.73 | 23.71 | 32.41 | 33.89 | 38.31 | 47 |
| | 14.05 | 14.34 | 24.13 | 32.63 | 34.85 | 36.63 | 48 |
| mean | 13.02 | 14.04 | 23.91 | 32.43 | 34.27 | 37.69 | 47.45 |
| Z-GLA-OH | 1.14 | 6.47 | 11.43 | 14.43 | 21.74 | 32.54 | 49 |
| | 1.44 | 7.66 | 11.9 | 14.26 | 21.93 | 32.61 | 49.4 |
| | 1.55 | 7.49 | 11.46 | 14.37 | 24.44 | 33.41 | 49.5 |
| mean | 1.38 | 7.21 | 11.60 | 14.35 | 22.70 | 32.85 | 49.30 |

Other compounds also performed well in the above in vitro test, including GPG-NH₂ and Z-GPG-NH₂ and compounds of related structure.

1. A method of treatment of an autoimmune or inflammatory disease or transplant rejection, the method comprising administering to a patient in need thereof a therapeutically effective amount of a compound, wherein said compound is a TPP II inhibitor.

2. A method as claimed in claim 1, wherein said compound is selected from formula (i) or is a pharmaceutically acceptable salt thereof:
R^{N1}R^{N2}N-A¹-A²-A³-CO—R^{C1} (i)
wherein A¹, A² and A³ are amino acid residues having the following definitions according to the standard one-letter abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine, R^{N1} and R^{N2} are each attached to the N terminus of the peptide, are the same or different, and are each independently
R^{N3},
(linker1)-R^{N3},
CO-(linker1)-R^{N3},
CO—O-(linker1)-R^{N3},
CO—N-((linker1)-R^{N3})R^{N4} or
SO₂-(linker1)-R^{N3},
(linker1) may be absent, i.e. a single bond, or CH₂ CH₂CH₂, CH₂CH₂CH₂, CH₂CH₂CH₂CH₂ or CH═CH,
R^{N3} and R^{N4} are the same or different and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two (same or different) optional substituents on R^{N3} and/or R^{N4} which may be:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N-, O- or S-acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
mono- or bicyclic C₁₋₁₀ heteroaryl;
non-aromatic C₁₋₁₀ heterocyclyl; or
halogen;
R^{C1} is attached to the C terminus of the tripeptide, and is:
O—R^{C2},
O-(linker2)—R^{C2},
N((linker2)R^{C2})R^{C3}, or
N(linker2)R^{C2}—NR^{C3}R^{C4},
(linker2) may be absent, i.e. a single bond, or C₁₋₆ alkyl or C₂₋₄ alkenyl, preferably a single bond or CH₂ CH₂CH₂, CH₂CH₂CH₂, CH₂CH₂CH₂CH₂ or CH═CH,
R^{C2}, R^{C3} and R^{C4} are the same or different, and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two (same or different) optional substituents on each of R^{C2} and/or R^{C3} and/or R^{C4} which may be one or more of:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N-, O- or S-acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
halogen;
mono- or bicyclic C₁₋₁₀ heteroaryl; or
non-aromatic C₁₋₁₀ heterocyclyl;

3. A method as claimed in claim 2 wherein said compound of formula (i) is such that:
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O-C₁₋₆ alkyl, O-C₁₋₆ alkyl-phenyl, NH—C₁₋₆ alkyl, or NH—C₁₋₆ alkyl-phenyl.

4. A method as claimed in claim 3, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

5. A method as claimed in claim 4, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine or 2-allylglycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O-C₁₋₆ alkyl, O-C₁₋₆ alkyl-phenyl, NH—C₁₋₆ alkyl, or NH—C₁₋₆ alkyl-phenyl.

6. A method as claimed in claim 5 wherein said compound of formula (i) is such that:
A¹ is G or A,
A² is L, I, or norleucine,
A³ is G or A,
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O-C₁₋₆ alkyl, O-C₁₋₆ alkyl-phenyl, NH—C₁₋₆ alkyl, or NH—C₁₋₆ alkyl-phenyl.

7. A method as claimed in claim 2 wherein
R^{N1} is hydrogen,
R^{N2} is hydrogen, C(═O)—OCH₂Ph or C(═O)—CH═CH-(2-furyl), and
R^{C1} is OH, O-C₁₋₆ alkyl, or NH—C₁₋₆ alkyl.

8. A method as claimed in claim 7 wherein said compound of formula (i) is
Z-GLA-OH, Bn-GLA-OH, FA-GLA-OH or H-GLA-OH.

9. A method as claimed in claim 8 wherein said compound of formula (i) is
Z-GLA-OH

10. A method as claimed in claim 2 wherein A¹ is selected from the group consisting of G, A and 2-aminobutyric acid.

11. (canceled)

12. A method as claimed in claim 2 wherein A² is selected from the group consisting of L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine and tert-butyl glycine.

13-16. (canceled)

17. A method as claimed in claim 2 wherein A³ is selected from the group consisting of G, A, V, P, 2-aminobutyric acid and norvaline.

18. (canceled)

19. A method as claimed in claim 2 wherein R^{N1} is hydrogen.

20. A method as claimed in claim 2 wherein R^{N2} is
R^{N3},
(linker1)-R^{N3},
CO-(linker1)-R^{N3}, or
CO—O-(linker1)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
saturated or unsaturated, branched or unbranched C₁₋₄ alkyl;
benzyl;
phenyl; or
monocyclic heteroaryl.

21. A method as claimed in claim 20 wherein R^{N2} is selected from the group consisting of hydrogen, benzyloxycarbonyl, benzyl, benzoyl, tert-butyloxycarbonyl, 9-fluorenylmethoxycarbonyl and FA.

22. (canceled)

23. A method as claimed in claim 2 wherein R^{C1} is:
O—R^{C2},
O-(linker2)—R^{C2}, or
NH-(linker2)R^{C2}
wherein
(linker2) may be absent, i.e. a single bond, C₁₋₆ alkyl or C₂₋₄ alkenyl, preferably a single bond or CH_{2,} CH₂CH₂, CH₂CH₂CH₂, CH₂CH₂CH₂CH₂ or CH═CH, and
R^{C2} is hydrogen or any of the following unsubstituted groups:
saturated or unsaturated, branched or unbranched C₁₋₅ alkyl;
benzyl;
phenyl; or
monocyclic C₁₋₁₀ heteroaryl.

24. A method as claimed in claim 23 wherein R^{C1} is selected from the group consisting of OH, O-C₁₋₆ alkyl, O-C₁₋₆ alkyl-phenyl, NH₂, NH—C₁₋₆ alkyl, and NH—C₁₋₆ alkyl-phenyl.

25-27. (canceled)

28. A method as claimed in claim 2 wherein said compound is selected from the group consisting of GPG-NH₂, Z-GPG-NH₂, Bn-GPG-NH₂, FA-GPG-NH₂, GPG-OH, Z-GPG-OH, Bn-GPG-OH, and FA-GPG-OH.

29. (canceled)

30. A method as claimed in claim 2 wherein said compound is selected from the group consisting of ALG-NH₂, Z-ALG-NH₂, Bn-ALG-NH₂, FA-ALG-NH₂, ALG-OH, Z-ALG-OH, Bn-ALG-OH, and FA-ALG-OH.

31. (canceled)

32. A method as claimed in claim 2 wherein A³ is not F, W, D, E or Y.

33. A method as claimed in claim 2 wherein A³ is not P.

34. (canceled)

35-39. (canceled)

40. A method of treatment of an autoimmune and/or inflammatory disease comprising administering to a patient in need thereof a therapeutically effective amount of a compound defined in claim 1.

41. A method of treatment as claimed in claim 1 wherein the condition is selected from Systemic Lupus Erythematosus, Rheumatoid Arthritis, Multiple Sclerosis, Sjögrens Syndrome, Diabetes Mellitus Type I or II, Psoriasis, Eczema, Ulcerous Colitis, and Chron's Disease.

42. A method of treatment of atheroschlerosis comprising administering to a patient in need thereof a therapeutically effective amount of a compound defined in claim 1.

43. A method of treatment of transplant rejection comprising administering to a patient in need thereof a therapeutically effective amount of a compound defined in claims 1.

44-48. (canceled)

49. A method for identifying a compound suitable for the treatment of an autoimmune or inflammatory disease or transplant rejection comprising contacting TPP II with a compound to be screened, and identifying whether the compound inhibits the activity of TPP II.

50. A method for identifying a compound suitable for the treatment of an autoimmune and/or inflammatory disease comprising contacting TPP II with a compound to be screened, and identifying whether the compound inhibits the activity of TPP II.

51. A method for identifying a compound suitable for the treatment of a condition selected from Systemic Lupus Erythematosus, Rheumatoid Arthritis, Multiple Sclerosis, Sjögrens Syndrome, Diabetes Mellitus Type I or II, Psoriasis, Eczema, Ulcerous Colitis, and Chron's Disease comprising contacting TPP II with a compound to be screened, and identifying whether the compound inhibits the activity of TPP II.

52. A method for identifying a compound suitable for the treatment of atheroschlerosis comprising contacting TPP II with a compound to be screened, and identifying whether the compound inhibits the activity of TPP II.

53. A method for identifying a compound suitable for the treatment of transplant rejection comprising contacting TPP II with a compound to be screened, and identifying whether the compound inhibits the activity of TPP II.
